(19) United States
(12) Patent Application Publication
(10) Pub. No.: US 2004/0208942 A1
(43) Pub. Date: Oct. 21, 2004

(54) Alpha lipoic acid based food supplement for increasing lean muscle mass and strength

(76) Inventors: Paul Gardiner, Mississaugu (CA); Derek Woodgate, Brampton (CA)

Correspondence Address: KENYON & KENYON, One Broadway, New York, NY, 10004, US

(21) Appl. No.: 10/841,404

(22) Filed: May 7, 2004

Related U.S. Application Data
(63) Continuation of application No. 09/775,431, filed on Feb. 1, 2001.
(60) Provisional application No. 60/178,957, filed on Feb. 1, 2000.

(51) Int. Cl. A61K 35/78; A61K 31/198; A61K 31/70; A61K 31/385

(52) U.S. Cl. 424/728; 514/440; 514/561; 514/565; 514/566; 514/23

(57) ABSTRACT

Food supplement compositions and their methods of use in increasing lean mass and/or muscle size and/or strength in individuals, particularly, athletes is described. The food supplements described comprise α lipoic acid or a derivative thereof, and an amino acid. Other food supplements described comprise a substance which can enhance and/or mimic activity of α lipoic acid, and a source of amino acid. The food supplement compositions described are suitable for supplementing the diet of an athlete and particularly for enhancing an athlete's muscle size and/or strength.

### CROSS-REFERENCE TO RELATED APPLICATIONS

This application claims the benefit of the U.S. provisional application Serial No. 60/178,957, filed Feb. 1, 2000, the content of which is incorporated herein by reference.

### FIELD OF INVENTION

The present invention is directed to food supplements which comprise α lipoic acid or a derivative thereof, and a source of amino acids or derivatives thereof; and to methods for supplementing the diet of an athlete and methods for enhancing an athlete's muscle size and/or strength, which methods employ these food supplements.

### BACKGROUND OF INVENTION

Food supplements based on natural products and approaches for enhancing an athlete's muscle size and/or strength have become popular exigencies in various sports and body building regimes. However, as athletes continually strive for improved performance, there is a continuing need for new more effective technologies to aid in increasing lean muscle mass, muscle size and/or strength.

### OBJECTS AND SUMMARY OF THE INVENTION

α lipoic acid (also known as alpha-lipoic acid, thioctic acid or 6,8-dithio octanoic acid and also referred to as lipoic acid) is a nutrient that the human body makes in minute quantities and may be obtained from yeast and liver. Studies have shown α lipoic acid can significantly increase the body's utilization of blood sugar in type II diabetes and that α lipoic acid may increase the metabolic clearance rate of glucose by 50% in type II diabetics. In Europe, α lipoic acid has been used as a substitute for insulin in the treatment of type II diabetes.

The present invention relates to supplementing the diet of an athlete with α lipoic acid or a derivative thereof in combination with a source of amino acids or derivatives thereof, may provide surprising enhancement of an athlete's muscle size and/or strength when administered to an athlete's diet. Accordingly, in one broad aspect the present invention provides new food supplements to increase the delivery and uptake of amino acids in the body. Preferably the invention provides food supplements particularly adapted for supplementing the diet of an athlete, preferably the food supplements of the present invention enhance an athlete's muscle size and/or strength.

According to one embodiment of the present invention there is provided a food supplement comprising α lipoic acid or a derivative thereof, and, a source of amino acids or derivatives thereof. Preferably the source of amino acids is whey protein or a derivative thereof.

According to another embodiment of the present invention, there is provided a food supplement comprising a substance which mimics and/or enhances activity of α lipoic acid or a derivative thereof, the supplement also containing glutamine or a derivative thereof. Preferably, where the substance enhances the activity of α lipoic acid or a derivative thereof, the supplement will contain both α lipoic acid or a derivative thereof and the enhancing substance.

According to another preferred embodiment, the supplements of the present invention comprise a α lipoic acid or a derivative thereof, and an amino acid or derivative thereof.

According to another preferred embodiment, the supplements of the present invention comprise a α lipoic acid or a derivative thereof, and glutamine or a derivative thereof.

In another broad aspect, the present invention provides methods of supplementing bodybuilding in an athlete comprising administering to the athlete an effective amount of a food supplement according to the present invention to achieve an increase in muscle size and/or strength.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The food supplements and methods of the present invention may provide further and significant increase in muscle size and/or strength enhancement or improvement in individuals as compared with supplements and methods employing only proteins and/or amino acids or α lipoic acid alone. While it is expected that the supplements and methods of the present invention will be of importance to bodybuilders and other athletes, the supplements and methods of the invention are not limited to those groups. Rather, any individual may use the supplements and methods of the invention. Indeed, the supplements and methods may have applications to all animals. As used herein, the term “animals” includes all members of the animal kingdom, preferably humans. As used throughout the present specification a reference to an increase in muscle size is also understood to mean an increase in lean muscle mass.

The food supplements described herein comprise the compounds specifically identified and suitable derivatives thereof, for example a salt or ester. Suitable salts include, but are not limited to, alkali and alkaline earth metal salts, for example sodium, potassium or calcium salts, while suitable esters include, but are not limited to, alkyl esters, for example, methyl, ethyl or propyl esters, or lactone esters.

As used herein “source of amino acid” means any peptide, polypeptides, protein or any composition of individual amino acids or individual amino acid. As will be readily appreciated by those skilled in the art, other sources of protein include milk protein, casein, any of the albumins including chicken egg albumin, and soy, may also be used as a source of amino acids.

Throughout the present specification, whey protein and a derivative thereof are identified as the preferred source of amino acids or protein. Commercially available whey protein derivatives include WPI 97, Whey Peptides, WPC 80, and ION EXCHANGE whey protein, although any form of whey protein isolates (WPI), whey peptides, whey protein concentrate (WPC), or whey protein isolated by ion exchange may be used. Additionally, hydrolyzed whey protein may be used.

Supplements

Although the present invention is not to be limited by any theoretical explanation, it is believed that insulin is a primary factor that stimulates the uptake of glucose and amino acids into muscle cells and that the α lipoic acid both mimics and enhances the actions of insulin in glucose and amino acid transport into muscle cells. Insulin has been clearly shown to increase amino acid uptake into skeletal muscle, inhibit protein degradation, and stimulate protein synthesis: the net effect is an increase in protein efficiency and utilization. There it is theorized that α lipoic acid which mimics and enhances insulin sensitivity can decrease protein degradation and increase amino acid uptake, protein synthesis, nitrogen retention, muscle size and/or strength.

Accordingly, in its broad aspect the present invention provides a food supplement comprising α lipoic acid or a derivative thereof in combination with a source of amino acids. According to one embodiment the source provides varied amino acids, preferably such source of amino acids is whey protein selected from the group consisting of WPI, Whey peptides, WPC, ion exchange whey protein, although as indicated above, other protein sources may be used.

According to another embodiment of the present invention, there is provided a food supplement comprising a substance which mimics and/or enhances the activity of α lipoic acid or a derivative thereof, the supplement also containing a source of amino acids. According to a preferred embodiment, a source of amino acids is preferably selected from the group consisting of WPI, whey peptides, WPC, hydrolyzed whey protein, ion exchange whey protein, or lactoferrin, although as indicated above, any other protein source may be used.

According to another preferred embodiment, the supplements of the present invention comprise a α lipoic acid or a derivative thereof, and an amino acid or derivative thereof. Any amino acid type may be used in this embodiment, however only the single form of amino acid or a derivative thereof is used. For example, the combination may include leucine, isoleucine, valine, arginine, or alanine or peptides like alanyl-glutamine, glutamine-glycine.

According to another embodiment of the present invention, there is provided a food supplement comprising a substance which mimics and/or enhances activity of α lipoic acid or a derivative thereof, the supplement also containing glutamine or a derivative thereof.

According to another embodiment, a food supplement of the present invention comprises α lipoic acid or a derivative thereof and glutamine or a derivative thereof.

As will be readily appreciated by those skilled in the art, the supplement is not limited to only one form of α lipoic acid or a derivative thereof and one source of amino acids. Indeed the present invention provides for combinations of substances and sources of amino acids, in differing amounts.

The food supplement compositions of the present invention may be provided in a variety of formats, for example, in liquid form, powder form, protein bar form or encapsulated in liposomes. Powders are preferable and are prepared to be suitable for mixing with water or other liquids. The food supplement compositions in powder or granular form may be provided in accordance with customary processing techniques, for example as spray dried powders, or the like.

The food supplement compositions can also contain ascorbic acid (vitamin C), for example in amounts equal to or exceeding the recommended minimum daily requirements. Another component for possible use in the food supplements of the present invention comprises beta-hydroxy, beta-methyl butyrate (HMB), in amounts known in the art. As will be appreciated by those skilled in the art, supplements may include one or more of a large number of other component which include, as non-limiting examples, carbohydrates, dextrose, vitamins, minerals, herbs, prohormones, ribose and lecithin.

The food supplement compositions may further comprise natural and/or artificial flavoring components, dyes or other coloring additives, preservatives and other conventional food supplement additives known in the art.

Methods of Use of Supplements

The food supplements according to the present invention may be employed in methods for supplementing the diet of an athlete, and/or for enhancing an athlete's muscle size and/or strength. The food supplement compositions of the present invention are particularly advantageous for creating an increased anabolic environment for obtaining extra growth in lean muscle mass and/or strength. Accordingly, the present invention provides methods of supplementing bodybuilding in an athlete comprising administering to the athlete an effective amount of a food supplement according to the present invention.

Administration of an effective amount of the supplements and substances of the present invention is defined as an amount effective, at dosages and for periods of time necessary to achieve the desired result. The effective amount of the supplements of the invention may vary according to factors such as the age, sex, and weight of the athlete. Dosage regime may be adjusted to provide the optimum response: Several divided doses may be administered daily or the dose may be proportionally reduced as indicated by the exigencies of an individual athlete's situation.

As will be readily appreciated a food supplement in accordance with the present invention may be administered in a single serving or in multiple servings spaced throughout the day. In a preferred embodiment, a food supplement in accordance with the present invention may be administered once in the morning, once immediately or shortly after training and once in the evening on a daily basis. As will be understood by those skilled in the art, servings need not be limited to daily administration, and may be on an every second or third day or other convenient effective basis. As well the administration on a given day may be in a single serving or in multiple servings spaced throughout the day depending upon the exigencies of the situation.

In order to maximize the effects of a food supplement according to the present invention, in enhancing muscle size and/or strength, it is preferred that the food supplement is administered to the diet of the athlete immediately following an exercise period. On non-workout days, the food supplement may be administered anytime during the day, although administering a first amount of a food supplement upon awakening or otherwise during the morning hours is preferred.

The embodiments set forth in the present application are provided only to illustrate various aspects of the invention and additional embodiments and advantages of the food supplements and methods of the present invention will be apparent to those skilled in the art.

### EXAMPLES

The present invention will be further explained in the following examples. However, the present invention should not be construed as limited as to scope and nature of the present invention. One of the ordinary skill in the art will understand how to vary the exemplified preparations to obtain the desired results.

### Example 1

In this example, an athlete consumes two servings of the food supplement as described herein daily, a serving of the food supplement immediately following his/her workout, and an additional serving 4 hours post workout. Each serving of the food supplement is about 100 grams and contains the following:

| Component | Amount | |
|---|---|---|
| α Lipoic acid | 200 | mg |
| Glutamine | 20 | g |
| Dextrose | 60 | g |
| Whey Protein | 30 | g |
| Arginine | 1 | g |
| Leucine | 1 | g |
| Phenylalanine | 1 | g |
| Methionine | 500 | mg |
| Glycosidal Saponins | 200 | mg |
| N-Acetyl cysteine | 100 | mg |
| Vitamin B6 | 10.5 | mg |
| Magnesium | 200 | mg |
| Vitamin E | 400 | IU |
| Potassium Phosphate | 100 | mg |
| Chromium Picolinate | 200 | mcg |

Each approximate 100 gram serving is mixed with about 15 ounces of cold water to provide a liquid drink. An additional 8 ounces of water may be consumed after the food supplement liquid drink is consumed.

### Example 2

In this example, an athlete consumes two servings of the food supplement as described herein daily, a serving of the food supplement immediately following his/her workout, and an additional serving 4 hours post workout. Each serving of the food supplement is about 120 grams and contains the following:

| Composition | Amount | |
|---|---|---|
| α Lipoic acid | 200 | mg |
| Glutamine | 10 | g |
| Dextrose | 75 | g |
| Whey Protein | 30 | g |
| Arginine | 1 | g |
| Leucine | 1 | g |
| Phenylalanine | 1 | g |
| Methionine | 500 | mg |
| Glycosidal Saponins | 200 | mg |
| N-Acetyl cysteine | 100 | mg |
| Vitamin B6 | 10.5 | mg |
| Magnesium | 200 | mg |
| Vitamin E | 400 | IU |
| Potassium Phosphate | 100 | mg |
| Chromium Picolinate | 200 | mcg |

Each approximate 100 gram serving is mixed with about 15 ounces of cold water to provide a liquid drink. An additional 8 ounces of water may be consumed after the food supplement liquid drink is consumed.

### Example 3

The muscle size and/or strength enhancing regime of Example 1 is modified so that the athlete consumes four servings of the food supplement daily, with each serving being approximately 55 g of the supplement and comprising:

| Component | Amount | |
|---|---|---|
| α Lipoic acid | 100 | mg |
| Glutamine | 10 | g |
| Dextrose | 30 | g |
| Whey Protein | 15 | g |
| Arginine | 0.5 | g |
| Leucine | 0.5 | g |
| Phenylalanine | 0.5 | g |
| Methionine | 250 | mg |
| Glycosidal Saponins | 100 | mg |
| N-Acetyl cysteine | 50 | mg |
| Vitamin B6 | 5.25 | mg |
| Magnesium | 100 | mg |
| Vitamin E | 200 | IU |
| Potassium Phosphate | 50 | mg |
| Chromium Picolinate | 100 | mcg |

Each supplement serving is mixed with 8 ounces of cold water to provide a liquid drink. An additional 8 ounces may be consumed after the food supplement liquid drink is consumed.

### Example 4

The dosage regime described in Example 1 is completed, on workout days. The serving is consumed immediately after exercise and then again 4 hours later. On non-exercising days the athlete consumes the food supplement as described in Example 2. The serving is consumed upon waking, prior to bed, and at two other time points during the day. Alternatively the supplement is combined with other liquid drinks or foods as desired. The regime is continued for a minimum of 8 weeks to enhance increases in muscle size and/or strength.

The servings set forth in these examples are designed for a 2500 calorie diet. Daily values can be increased or decreased depending on the needs of the individual athlete.

The examples and embodiments set forth in the present applications are provided only to illustrate various aspects of the invention and additional embodiments and advantages of the food supplements and method of the present invention will be apparent to those skilled in the art.

1. A food supplement, comprising alpha lipoic acid or a derivative thereof, and at least one ingredient selected from the group consisting of amino acids and derivatives thereof and sources of amino acids, in amounts effective to increase lean muscle mass and strength.

2-37 (Canceled).
